(19) United States
(12) Patent Application Publication
(10) Pub. No.: US 2026/0347763 A1
(43) Pub. Date: Nov. 5, 2026

(54) TRANSPARENT SUPPORT MEDIUM FOR 3D PRINTING

(71) Applicant: Ramot at Tel-Aviv University Ltd., Tel-Aviv (IL)

(72) Inventors: Tal DVIR, Tel Aviv (IL); Assaf SHAPIRA, Tel Aviv (IL)

(73) Assignee: Ramot at Tel-Aviv University Ltd., Tel-Aviv (IL)

(21) Appl. No.: 19/113,014

(22) PCT Filed: Sep. 27, 2023
(86) PCT No.: PCT/IL2023/051038

§ 371 Date: Mar. 19, 2025

Related U.S. Application Data
(60) Provisional application No. 63/410,274, filed on Sep. 27, 2022.

(51) Int. Cl. C12N 5/00; A61L 27/26; A61L 27/38; A61L 27/52; B33Y 70/10; B33Y 80/00

(52) U.S. Cl. CPC C12N 5/0062; A61L 27/26; A61L 27/38; A61L 27/52; B33Y 70/10; B33Y 80/00; C12N 2513/00; C12N 2533/20; C12N 2533/70; C12N 2533/74; C12N 2539/00

(57) ABSTRACT

A 3D-printing support medium is provided herein, comprising hybrid calcium-alginate hydrogel particles and a dispersion medium, formulated by combining calcium-alginate and a soluble polymer distinct from alginate, and the dispersion medium contains a minimum of 2 mM gluconate. These hybrid hydrogel particles possess an average size ranging from 0.1 μm to 50 μm, with exceptional homogeneity characterized by a particle size distribution of less than 50% RSD, resulting in a substantially transparent medium to visible light. The support medium is provided in various forms, including drained particles and slurries with different volume ratios of hydrogel particles to the dispersion medium. The process of preparing this support medium involves forming, pulverizing, washing, and contacting the hybrid hydrogel particles with a gluconate solution, ensuring the presence of gluconate within the hydrogel matrix.

### RELATED APPLICATIONS

This application claims the benefit of priority of U.S. Provisional Patent Application No. 63/410,274 filed on 27 Sep. 2022, the contents of which are incorporated herein by reference in their entirety.

### FIELD AND BACKGROUND OF THE INVENTION

The present invention, in some embodiments thereof, relates to 3D printing of biomaterials, and more particularly, but not exclusively, to a support medium for printing biomaterials based on transparent, heat-stable and biocompatible hydrogel particles.

Tissue engineering (TE) is a field of science that integrates knowledge from biology, materials sciences, engineering and medicine to develop artificial, functional tissue constructs to replace or support defected tissues. Rather than simply introducing cells into the diseased area to repopulate the injured tissue and restore function, tissue engineering involves the seeding of cells in or onto 3-dimensional (3D) biomaterials prior to transplantation. These materials serve as temporary scaffolds supporting the cells and promoting their reorganization to a functional tissue. Following implantation and full integration in the host, the scaffold degrades, leaving a functional tissue patch on the defected organ. In recent years it has been recognized that effective organization of cells into tissues with morphological and physiological features resembling those in vivo requires a 3D scaffold that precisely mimic the biochemical, structural and mechanical properties of the natural tissue's extracellular matrix (ECM).

Thus, researchers have initially focused on developing materials and technological tools to recapitulate aspects of this specialized microenvironment. While synthetic scaffolds made of biocompatible materials can be fabricated to closely mimic the ECM structure, they still lack much of the fine, complex architecture and biochemical cues that can be found in the native ECM. Contrary, ECM and ECM derived materials can be processed, manipulated and fabricated to create a 3D scaffold that reliably recapitulate the natural cell microenvironment.

One of the most promising methodologies for fabrication of complex, artificial tissue patches is the emerging “bottom-up” or “modular” approach. This method is based on the generation of “modules”, namely microscale tissue building blocks that incorporate a complex artificial micro and nano architecture that resemble to that of a native tissue. The modules can be fabricated by using various methods such as cell printing, self-assembled cell aggregates, generation of cell sheets and fabrication of cell-laden hydrogels. These building blocks are than assembled to form a large tissue construct using methods like random packing, stacking of layers and 3D bioprinting.

Three-dimensional (3D) printing is a technology that allows bottom-up construction of complex structures. The boundaries of the printed model are defined by a computer-aided design (CAD) software and accordingly the printer deposits the material in a layer by layer manner. Recent advances in the field have enabled utilization of various printing technologies for delivering living cells with materials. Although in its infancy, one of the promising technologies to print tissues is by micro-extrusion. Compared to inkjet and laser-assisted printing, which deposit dissociated liquid droplets, extrusion printers use robotically controlled extrusion heads to deposit continues strands of materials in which cells can be incorporated. Up to date such printers have been used to print aortic valves and branched vascular trees. However, printing complex tissues such as the myocardium, which consists of various cell types (cardiac fibroblasts and myocytes) together with a dense vasculature, remained a challenge. One of the main reasons for that is the relatively inferior physical properties of biomaterials such as natural, ECM derived substances that are being used as biocompatible and biodegradable “bio-inks” for the printing process. In contrast to synthetic polymers, fabrication of thick, multilayered complex structures with the abovementioned substances results in an unstable structure with low shape fidelity when executed using conventional micro-extrusion 3D printing methods. A suitable temporary support that holds the extruded material and stabilizes the delicate printed structure until it is fully cured is a possible solution to this problem.

WO 2015/017421 discloses structure material that comprises a fluid that transitions to a solid or semi-solid state after deposition of the structure material, wherein the support material comprises material comprises a gel material, a hydrogel material, μm-sized particulates and/or a thermo-reversible material. WO 2015/017421 appears to disclose a method for fabricating a structure such as a biological tissue or a tissue engineering scaffold using 3D printing, where the printing method comprises a support bath within which the tissue scaffold is fabricated and which provides divalent cations for crosslinking the printed material. Further, use of a cross-linker concentration in a method for producing rapid prototyping is discussed in EP1517778B; while DE102012100859A discloses a method for producing and printing a 3D structure containing living cells, which may comprise of printing in a high density liquid.

WO 2016/040095 discloses support matrix for 3D printing, comprising a graphene aerogel or a gelled ionic liquid, wherein the gelled ionic liquid comprises an ionic liquid gelled with fumed silica, precipitated silica, chalk, carbon black, paraffin composition, silicone oil, or any combination thereof.

U.S. Patent Application Publication No. 20180057682 discloses an organic microgel system as support material for 3D printing of soft materials such as silicone and methods for manufacturing and using the organic microgel system. According to this document, the organic microgel system comprises a plurality of microgel particles formed by blending a di-block copolymer and a tri-block copolymer in an organic solvent, thereby forming an organic microgel system for high precision 3D printing of silicone objects with complex shapes.

WO 2019/234738 to the present inventors provides a see-through transparent, heat-stable, biocompatible and biodegradable hydrogel-based particulate support medium, made of calcium alginate particles and at least traces of a soluble polymer, such as xanthan gum. The hydrogel-based particulate support medium are homogeneous calcium alginate hydrogel particles, or hybrid hydrogel particles, having an average size that ranges from 0.1 μM to 5 μM, and a particle size distribution of less than about 20% RSD.

Additional background art includes U.S. Patent Application Publication Nos. 2015057786 and 20210252777, U.S. Pat. No. 11,124,644, WO 2017/081040, WO 2016/182969, WO 2014/194180, WO 2014/194180, EP 1517778 and DE 102012100859, and Aarstad et al., Polymers, 2017; Bhattacharjee et al., Sci. Adv., 2015; He et al., Sci. Reports., 2016; Hinton et al., ACS Biomater. Sci. Eng., 2016; Hinton et al., Sci. Adv., 2015; Kuo et al., Biomaterials, 2001; and O'Bryan et al., Sci. Adv., 2017.

### SUMMARY OF THE INVENTION

To improve the optical clarity of calcium-alginate based granular support medium, such as disclosed in WO 2019/234738, which is incorporated herein in its entirety as if fully disclosed herewith, a calcium alginate hydrogel was prepared, crushed (pulverized) and washed to obtain a slurry of particulate hydrogel, and thereafter sodium D-gluconate was introduced to the washed particulate hydrogel slurry, prior to the optional addition of a non-alginate soluble polymer. After extensive tests and calibrations with a diversity of soluble compounds and different calcium sequestrants, the present inventors have found that optimal result could be attained when sodium D-gluconate is added to the support medium to reach a final concentration of, for example, at least 2-5 mM (2-5 millimolar). This was achieved by mixing the compacted pellet of washed calcium-alginate hybrid hydrogel particles, after the final centrifugation step, with an aqueous solution containing sodium D-gluconate. If needed as a commercial product, the resulting slurry can be re-centrifuged and drained to yield a more compact pellet. Soluble polymers, such as xanthan gum, may then be added (or reintroduced) into the mixture as previously described.

In contrast to the formulations that have been previously developed by others, the support medium presented herein provides a unique combination of features, all of which are desirable for efficient and accurate 3D printing using bioinks that contain live cells, and using ECM-derived bioinks in particular. The support media is both transparent, biocompatible (and made of non-animal origin materials), cell-friendly, and stable at temperatures higher than 37 °C, allows free-form printing and curing in a wide range of temperatures, and extraction by a controllable, non-mechanical delicate process.

Hence, according to an aspect of some embodiments of the present invention there is provided 3D-printing support medium, which includes a plurality of hybrid hydrogel particles and a dispersion medium, whereas the hybrid hydrogel includes calcium-alginate and a non-alginate soluble polymer, wherein the dispersion medium includes at least 2 mM gluconate. The hybrid hydrogel consists of a polymeric network and a dispersion medium, whereas the polymeric network includes calcium-alginate and possibly some calcium-alginate-network incorporated non-alginate soluble polymer, and the dispersion medium may also include the non-alginate soluble polymer.

In some embodiments, the particles are characterized by an average size that ranges from 0.1 μm to 50 μm, and a homogeneity characterized by a particle size distribution of less than 50% RSD.

In some embodiments, the support medium is characterized by being substantially transparent to visible light.

In some embodiments, the gluconate is an anion of a non-calcium gluconate salt. In some embodiments, the non-calcium gluconate salt is sodium gluconate, potassium gluconate, zinc gluconate, magnesium gluconate, and any combination thereof.

In some embodiments, the soluble polymer is xanthan gum.

In some embodiments, the support medium includes xanthan gum at a concentration that ranges from 0.0001 to 4% w/w or % w/v of the total weight or volume of the support medium.

In some embodiments, the support medium provided herein may be in the form a plurality of drained hybrid hydrogel particles.

In some embodiments, the support medium provided herein in a form of a slurry, wherein a volume ratio of the plurality of hybrid hydrogel particles to an excess of the dispersion medium ranges from 1:0.001 to 1:3.

According to another aspect of some embodiments of the present invention, there is provided a process of preparing the support medium provided herein, which is effected by:
i. forming a calcium-alginate hydrogel in the presence of the non-alginate soluble polymer, thereby affording a hybrid hydrogel;
ii. pulverizing the hybrid hydrogel to thereby obtain a plurality of hybrid hydrogel particles;
iii. washing the plurality of hybrid hydrogel particles to thereby obtain a plurality of washed hybrid hydrogel particles; and
iv. contacting the washed hybrid hydrogel particles with a solution of gluconate.

In some embodiments, the process further includes:
v. draining the hybrid hydrogel particles from excess of the gluconate solution to thereby obtain drained hybrid hydrogel particles having gluconate therein.

In some embodiments, the process further includes:
vi. contacting the drained hybrid hydrogel particles or contacting the non-drained hydrogel particles with a solution of the non-alginate soluble polymer.

In some embodiments, the solution of the gluconate includes sodium gluconate at a concentration of at least 2 mM.

In some embodiments, forming the calcium-alginate hydrogel is effected in the presence of an insoluble calcium salt and an acidifier.

In some embodiments, the acidifier is a glucono-S-lactone.

In some embodiments, the insoluble calcium salt is calcium carbonate.

In some embodiments, the concentration of the non-alginate soluble during the formation of the calcium-alginate hydrogel ranges 0.001-20% (w/v).

In some embodiments, the support medium is washed/treated with a solution the includes the soluble non-alginate polymer at a concentration that ranges 0.0001-4% (w/v).

In some embodiments, the soluble polymer is xanthan gum.

According to another aspect of some embodiments of the present invention, there is provided a selling unit includes the support medium provided herein.

In some embodiments, the support medium is sterile and/or detoxified.

In some embodiments, the support medium is ready for use without further dilution.

In some embodiments, the support medium is in the form of a drained slurry, or a drained plurality of the hybrid hydrogel provided herein.

In some embodiments, the selling unit further includes a dilution solution.

In some embodiments, the selling unit is packaged in a packaging material and identified in print on or in the packaging material, for use as a support medium in 3D printing process and/or printer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying images. With specific reference now to the images in detail, it is stressed that the particulars shown are by way of example and for purposes of illustrative discussion of embodiments of the invention. In this regard, the description taken with the images makes apparent to those skilled in the art how embodiments of the invention may be practiced.

In the images:

FIG. 1 presents a flow-chat of a simplified process for preparing the gluconate-containing support medium provided herein, showing the preparation steps of four exemplary embodiments thereof;

FIG. 2. presents the results of a comparative turbidity test conducted for the support medium following treatment with each of the abovementioned supplements, whereas the support media samples were supplemented with 5:1 (w/w or v/v) of the following solutions: HEPES buffered saline (pH 7.4) with 1.5 mM CaCl₂) (“HEPES B.S”); Dulbecco's Phosphate Buffered Saline, devoid of calcium and magnesium (“PBS”); 150 mM sodium D-gluconate (“D-Gluc”), and the results are given in Nephelometric Turbidity Units (NTU);

FIG. 3 is a black and white photograph that compares the transparency of the support medium following treatment with DMEM/F-12 cell culture medium (left) or 150 mM sodium D-gluconate (right);

FIGS. 4A-B present the result of the cell viability assays, as determined 48 hours post-seeding of cells in growth media supplemented with 5:1 V/V ratio PBS (a positive control of a known non-toxic material), 5:1 V/V ratio 150 mM sodium D-gluconate solution and 333:1 V/V ratio ProClin™ 915 (a negative control of a known toxic material), wherein FIG. 4A shows fluorescence as a function of cell growth (quantified using PrestoBlue™ reagent), and FIG. 4B shows microscopic images of cells;

FIGS. 5A-B present light-microscopy images, taken with 4× objective lens (scale bars=200 micrometer), showing the effect of sodium D-gluconate on printing quality, whereas FIG. 5A is a pattern printed as a control experiment using support medium supplemented with 5:1 V/V ratio saline, and FIG. 5B is the same pattern printed using support medium supplemented with 5:1 V/V ratio 150 mM sodium D-gluconate solution; and

FIG. 6 presents a comparative chart, showing the turbidity values measured for support media prepared without gluconate (“Saline treatment”), supplemented with another biocompatible calcium chelator (“PBS treatment”), with sodium gluconate added during the initial gel preparation and wetted with saline (“D-Gluc in gel prep+Saline treatment”), and with sodium D-gluconate solution after preparation (“D-Gluc treatment”).

### DESCRIPTION OF SOME SPECIFIC EMBODIMENTS OF THE INVENTION

The present invention, in some embodiments thereof, relates to 3D printing of biomaterials, and more particularly, but not exclusively, to a support medium for printing biomaterials based on transparent, heat-stable and biocompatible hydrogel particles.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

On the path to realizing the ultimate goal of fully customized engineering of human organs, this invention offers a solution to enable this achievement. It provides the necessary support materials for 3D printing intricate volumetric structures required for this purpose. 3D-printing of volumetric structures made of ECM-derived bioinks involves a biopsy of tissue from a patient, after-which the cellular and a-cellular materials are separated. While the cells are reprogrammed to become pluripotent stem cells, the ECM is processed into a personalized, temperature-sensitive hydrogel that can be used as a “bioink” for 3D printing. Following mixture of the cells and the hydrogel to generate cellularized bioinks, the printed cells efficiently differentiate to create patient-specific, immunocompatible tissues. However, printing of these tissues is limited to relatively thin structures with simple geometry, resulting from the delicate nature of the ECM-derived bioinks that tends to collapse in its uncured state. Thus, a printing strategy has been developed and disclosed herein, aiming to maintain the shape of the printout until the bioinks are fully cured. This strategy is based on the provision of a particular support medium that can be specifically tailored to support the unique features of the ECM-derived bioink formulation. Apart of being transparent (an advantage over the use of other types of support such as those comprised of gelatin particles), the presently disclosed support medium is stable at elevated temperature that is required for optimal curing of many ECM-based building materials.

In order to improve the ability to print stable, complex, multilayered structures composed of biomaterials such as processed ECM and/or ECM derived substances at high resolution, the present inventors developed a fabrication method based on fused-deposition modeling within a unique supportive material (WO 2019/234738). At the basis of this method is a special formulation of a hybrid hydrogel composed of calcium alginate (the structural component of the hydrogel) and xanthan gum (a washable additive used to destabilize the calcium alginate gel, making it amenable for further processing, while also improving its printing supporting capacity). This granular media provided in WO 2019/234738 supports the printed material and preserve its shape during the fabrication process and the following curing phase. When fully cured, the biocompatible, biodegradable, heat-stable, non-animal based support can be degraded, releasing the stable, self-supported 3D-printed structure. A key feature of this supporting media is its transparency and fluidity. This quality is especially beneficial in cases when the user needs to inspect the printed construct while fabricated and modify printing parameters accordingly without interrupting the procedure. Furthermore, the fact that light can freely penetrate the support enable an efficient use of photocurable materials as “inks”.

In the present invention, the inventors developed a method for further improving the optical clarity of the support, which is especially critical when printing is performed in large volumes of support through which light needs to penetrate.

The present invention provides a significantly improved support medium compared to the support medium described in WO 2019/234738. The herein-provided support medium that allows stable 3D printing of complex, multilayered structures composed, inter alia, of biomaterials, such as processed ECM and/or ECM derived substances at high resolution, using a fabrication method based on fused-deposition modeling within the unique support medium. The improved see-through transparency of the support medium provided herein to visible light further facilitates the monitoring of the printing process at real time: using the presently provided support medium, the user will have better ability to inspect the printed construct while fabricated and modify printing parameters accordingly without interrupting the procedure. The improved see-through transparency of the media also improves automatic calibration and monitoring of the printing process by 3D printers equipped with compatible optical sensors. This enables the user to calibrate and tune the printer more conveniently and accurately, resulting in a better degree of printout refinement. Finally, the improved transparency of the support media increases the curing efficiency of light-curable printed materials by external illumination.

From a clinical point of view, the present invention promotes high-resolution fabrication of complex cellular or acellular scaffolds composed of biomaterials such as processed ECM or ECM-derived substances for tissue engineering and regenerative medicine. From a research point of view, the present invention further promotes the development of new biomaterials with desirable features which behavior during printing could be directly monitored by virtue of the excellent transparency of the support media. Industrially, the present invention promotes the fabrication of high-resolution 3D printing of materials that cure under elevated temperature and require a delicate procedure for resolution from the support.

As a strategy to improve the transparency of the support, the present inventors sought to decrease the availability of calcium ions to the alginate chains in the hydrogel, resulting in a less dense and more ordered polymer network. It should be noted, however, that calcium ions serve as crosslinkers that link alginate chains together to create a physical hydrogel. Thus, the introduction of potent chelators, such as citrate, EDTA, EGTA, and polyphosphates, which bind calcium ions with high affinity, resulted in a rapid destabilization of the hydrogel and the loss of its supporting capability. Moreover, these calcium-chelating agents have the potential to adversely affect cells through a direct cytotoxic activity or by depravation of calcium that is essential for cell metabolism and activity. Considering the above, the present inventors contemplated non-toxic substances that act to sequester calcium ions thus resulting in a less dense and more ordered calcium-alginate hydrogel on one hand, while preserving the integrity of the hydrogel and its supporting capability on the other hand.

Glucono-delta-lactone (GDL; glucono-δ-lactone), also known as gluconolactone, typically used as a sequestrant and an acidifier, is a lactone of D-gluconic acid. It is often used as an acidulant and sequestrant in the food industry. This white, odorless crystalline powder that is water-soluble, is commonly utilized in food processing as a slow-acting leavening agent, a coagulant for tofu production, and as a pH control agent in various food products. It is considered a safe food additive and is known for its ability to gradually lower the pH of a food product, making it a versatile ingredient in food manufacturing. Upon addition to water, GDL partially hydrolyzes to gluconic acid as shown below, with the balance between the lactone form and the acid form established as a chemical equilibrium, whereas the rate of hydrolysis is increased by heat and alkalinity (high pH).

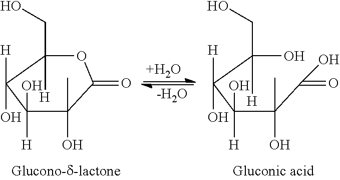

Calcium gluconate ([CH₂OH(CHOH)₄COO]₂Ca, CAS Reg. No. 299-28-5) is a water-soluble calcium salt which is affirmed as GRAS, and used as a nutritional additive and a medicine to treat hypocalcemia. It is the calcium salt of gluconic acid which may be produced by neutralization of gluconic acid with lime or calcium carbonate. It is also used as a calcium source for generation of calcium-alginate hydrogels, in which the gluconate ion acts as a calcium sequestrant that compete with the alginate on calcium binding. This competition retards calcium binding by the alginate chains, slowing down the rate of crosslinking. The result is more homogenous and transparent gels when compared to these prepared with solution of other calcium salts in which the availability of the calcium ion is much higher (like calcium chloride).

While the strategy of using calcium sequestrants in the gelation process has been suggested in the art, there are no documented attempts to use such molecules for improving the optical qualities of already prepared gels—and indeed, there are no reports of use of gluconate post-gelation hydrogels. While reducing the present invention to practice, the inventors considered that while not potent enough to destabilize the gel, weak sequestrants may be able to render a pre-formed hydrogel more transparent.

Without being bound by any particular theory, it is assumed that the effect of gluconate on a pre-formed hydrogel is by transforming the hydrogel into a less dense, more ordered polymer network by reversibly sequestering calcium ions that are bound to the alginate chains with a reduced affinity. These might be disordered calcium cations such as those that contribute to further lateral interaction between alginate chain dimers in the junction zone, as suggested in the literature, and/or calcium cations loosely bound to alginate MM or MG blocks. The inventors have theorized that there are distinct “sub-mechanisms” for the two processes: when using gluconate in its calcium salt form (2:1 gluconate to calcium molar ratio) in the gel formation step, the rule of the gluconate ion is to slow down the rate of alginate ionic crosslinking, by retarding the uptake of calcium by the alginate chains. This slower rate of gel formation results in a more ordered (crystallized) gel that is characterized by higher transparency in comparison to gels crosslinked by calcium chloride (in which the calcium is more available). Contrary, when using gluconate (not in its calcium form), on a pre-formed gel, where the ratio of gluconate to calcium in the buffer is high (for example, 25 mM gluconate vs. 1.5 mM calcium), the mechanism leans more towards “refining” the gel, taking away excess, low affinity bound calcium that impairs the gel's ordered form.

The formulation of calcium alginate-xanthan gum hybrid hydrogel, described in WO 2019/234738, which is further processed into a calcium alginate hydrogel-based fine and uniform particulate media, was found suitable for 3D-printing support media. In WO 2019/234738, the present inventors use a water-insoluble calcium carbonate salt (CaCO₃) to retard the gelation process, and further use GDL as an acidifier to slowly solubilize CaCO₃ and release calcium ions for alginate gelation; in WO 2019/234738 GDL is thereafter washed-out, together with unbound calcium, from the hydrogel during the washing steps and prior to re-introduction of a soluble polymer.

As used herein, the term “hydrogel” refers to a three-dimensional network or matrix immersed in a dispersion medium and composed of hydrophilic polymer chains that are crosslinked, entangled or otherwise bound to each other. Typical to hydrogels, the crosslinked polymer chains form a network that is capable of absorbing and retaining a substantial amount of the dispersion medium within their structure. The dispersion medium is the liquid that is dispersed throughout the hydrogel network, and therefore the dispersion medium regarded as an integral part of a hydrogel; the hydrogel is never completely dry, as it will no longer be defined as a hydrogel without the dispersion medium; however, in some embodiments of the present invention, the hydrogel may be dried and rehydrated, namely the drying may be reversible by adding the dispersion medium to the dried crosslinked polymer.

The dispersion medium, which is essentially an aqueous solution, plays an important role in determining the properties of the hydrogel. For example, the viscosity of the dispersion medium will affect the flowability of the hydrogel. The polarity of the dispersion medium will affect the interactions between the polymer chains and the dispersion medium. The ionic strength of the dispersion medium will affect the cross-linking of the polymer chains. In the case of a calcium-alginate hydrogel, the hydrophilic polymer is typically sodium alginate, and the dispersion medium is aqueous, comprising water and may include other soluble substances as required by the specific application. The crosslinking of sodium alginate polymer chains in the presence of calcium ions forms a stable and biocompatible hydrogel that exhibits the capacity to swell, gelate, and maintain its structural integrity in the presence of the liquid medium, making it suitable for various medical, pharmaceutical, and biomedical applications.

In the context of the present invention, the dispersion medium can be replaced by soaking and/or washing the pre-formed hydrogel or a plurality of particles thereof. In the context of the present invention, in any of the particles-treatment steps, the dispersion medium within and around the hydrogel particles may be a subject to manipulation in terms of composition and volume.

The hydrogel of the support medium provided herein is a hybrid hydrogel due to the presence of a non-alginate soluble polymer during gelation (hydrogel formation) as part of the gelation solution, or the addition of a non-alginate soluble polymer to a pre-formed calcium-alginate hydrogel.

In the context of the present invention, the combination of two or more polymers in the formation and/or processing of a hydrogel is referred to herein as a “hybrid hydrogel”. Thus, in the context of some embodiments of the present invention, the term “hybrid hydrogel” refers to a hydrogel or a processed hydrogel preparation that comprises at least two types of polymers, a first polymer that is a hydrogel-forming polymer and a second polymer that is a soluble polymer, whereas the hybridization is effected either by co-jellification or by pre- or in-(pulverizing/grinding) processing introduction or by post-washing introduction/re-introduction a second (or more) polymer into the formed hydrogel network and/or to it's the dispersion medium. The term “hybrid hydrogel”, as used herein, is meant to encompass all forms of possible interactions between non-alginate soluble polymer molecules (e.g., xanthan gum) and the crosslinked calcium-alginate network (e.g., calcium alginate hydrogel), including it being entangled or mechanically bound/entrapped/caged, or as a solute in the dispersion medium. In the context of embodiments of the present invention, the term “hybrid hydrogel” refers to calcium-alginate hydrogel that has been prepared and/or processed in the presence of a non-alginate soluble polymer, namely a soluble polymer that is not alginate, regardless of whether the formed hydrogel had been further processed, such as, for example, crashed, washed, or treated with other ingredients. In the context of some embodiments of the present invention, unless specified otherwise, all reference to “calcium-alginate hydrogel”, or “hydrogel”, is a reference to a hybrid hydrogel as defined hereinabove.

By entangled or otherwise bound it is meant that some of the non-alginate soluble polymer is non-covalently bound to the alginate strands, but rather becomes entangled in the calcium-alginate network; beyond mechanical entanglement, it is assumed that other non-covalent bonding (H-bonds between two oligosaccharides in aqueous environment) keeps the hybrid hydrogel stable under repeated washing; thus, the incorporation of some of the non-alginate soluble polymer is substantially irreversible unless the network itself is disrupted. The hydrogel network may be disrupted by, e.g., enzymatic digestion of the alginate polymer or extraction of the crosslinking calcium ions from the network by strong chelators, in which case the non-alginate soluble polymer which has been trapped in the network will be released.

It is noted herein that the concentration of the non-alginate soluble polymer molecules that is incorporated into the polymeric network of the formed hybrid hydrogel, or the relative amount thereof with respect to the amount of alginate, correlates to the concentration of the both the alginate and the non-alginate soluble polymer in the original dispersion medium in which the initial formation of the hybrid hydrogel takes place. However, since some of the polymer strands of both the alginate and the non-alginate soluble polymer may remain non-associated with the polymeric network after its formation, and may stay soluble in the dispersion medium, it is noted herein that in order to measure the exact composition of the hybrid hydrogel after washing, at least with respect to the alginate and the non-alginate soluble polymer, the hybrid hydrogel should be subjected to advanced analytical methods as described herein, which are mostly destructive to the hydrogel.

As opposed to the concentration of the non-alginate soluble polymer in the dispersion medium, the amount of it that is bound and incorporated into the alginate network is substantially invariable once the hybrid hydrogel is formed. Hence, when referring to the amount of the non-alginate soluble polymer in the support medium provided herein, it is noteworthy to consider two types of participation of the non-alginate soluble polymer in the final product: one type of participation is as an integral part of the network, which is regarded as essentially determined by its concentration and the alginate concentration in the dispersion medium of the initial hydrogel formation, and second as a solute in the dispersion medium, which can be manipulated by washing/treating the post-formation hydrogel.

While the concentration of the non-alginate soluble polymer in the dispersion medium can be set or determined by standard methods, the amount of the network-bound non-alginate soluble polymer is regarded as substantially fixed during various manufacturing and processing steps and usage of the support medium. Determining the amount of the network-bound non-alginate soluble polymer requires more advanced methods, such as NMR spectroscopy, FTIR, monosaccharide analysis following polysaccharide isolation and depolymerization, enzymatic fingerprinting, as well as polymer-specific antibodies. In embodiments wherein the non-alginate soluble polymer is from a biological source, indirect methods for assessing the concentration of the network-bound non-alginate soluble polymer in the hydrogel particles may be carried out using polymerase chain reaction (PCR) with primers that are specific for the biological source thereof.

The support media provided herein is characterized by advantageous heat and mechanical stability, as well as advantageous see-through transparency. While transparent calcium alginate hydrogels, as well as particles of such hydrogels are known in the art, it is the combination of alginate, calcium, gluconate and an additional water soluble and biocompatible polymer, such as xanthan gum, that render the herein-provided support medium for 3D printing advantageous over those known in the art. The combination of two or more polymers in the formation and/or processing of a hydrogel is referred to herein as a “hybrid hydrogel”. Thus, in the context of the present invention, the term “hybrid hydrogel” refers to a hydrogel or a processed hydrogel preparation that comprises at least two types of polymers (at least one of them can form a hydrogel and the other is a different polymer), either by co-jellification or by pre- or in-(pulverizing/grinding) processing introduction or by post-washing introduction/re-introduction a second (or more) polymer into the formed hydrogel network and/or to its surroundings.

In contrast to WO 2019/234738, the present invention provides an improvement of that support media, by further controlling the extent and density of crosslinking in the hydrogel, effected by treating the pre-formed, pulverized and washed calcium-alginate hydrogel with gluconate via the dispersion medium, prior to the optional reintroduction of xanthan gum, bringing the concentration of gluconate in the dispersion medium to a predetermined concentration (e.g., 2-5 mM or more).

It is noted that since gluconate, the anion of the salt of gluconic acid, is introduced into the support medium via the dispersion medium for its mild calcium-sequestrant properties, the gluconate that is introduced to the formed hybrid hydrogel is a non-calcium gluconate salt. Non-calcium gluconate salts include, without limitation, sodium gluconate, potassium gluconate, zinc gluconate, or magnesium gluconate. In some embodiments, the non-calcium gluconate salt is sodium gluconate.

### A Process of Preparing Hydrogel-Based Particulate Support Media:

According to some preferred embodiments of the present invention, the hybrid hydrogel is prepared by co-jellifying calcium ions, alginate and a non-alginate soluble polymer, such as xanthan gum, essentially as described herein and in the Examples section that follows below. It is noted however, that the hybrid hydrogel may also be prepared by forming the calcium alginate hydrogel, and adding the non-alginate soluble polymer during the processing of the calcium-alginate hydrogel. Without being bound by any particular theory, it is assumed that the addition of a non-alginate soluble polymer weakens the structure of the calcium-alginate hydrogel, as well as facilitating and preserving the homogeneity of the particulate material following pulverizing; hence, while pulverization and homogenization of a calcium alginate hydrogel typically results in large, bulky and irregular flakes and/or aggregates that constitute an inferior support medium, the calcium alginate-xanthan gum hybrid hydrogel can be processed to afford fine, homogenous calcium alginate hydrogel particles.

Thus, according to an aspect of some embodiments of the present invention, there is provided a process of preparing the support medium provided herein, which is effected by:
i. forming a calcium-alginate hydrogel in the presence of a non-alginate soluble polymer, thereby forming a hybrid hydrogel;
ii. grinding the hybrid hydrogel to thereby obtain a plurality of hydrogel particles;
iii. washing the hybrid hydrogel particles to thereby obtain a plurality of washed hydrogel particles;
iv. contacting the washed hydrogel particles with a solution of gluconate; and
v. optionally draining the hydrogel particles from excess of the solution of the gluconate to thereby obtain drained hydrogel particles having gluconate therein, which can be used as the support medium provided herein; and/or
vi. optionally contacting the drained/non-drained hydrogel particles with a solution of the non-alginate soluble polymer, to thereby obtaining the support medium.

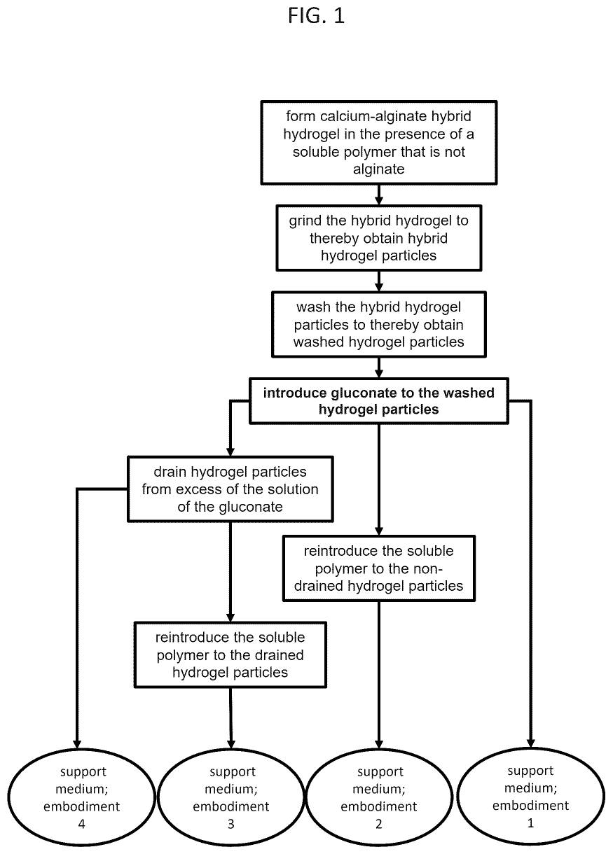

FIG. 1 presents a flow-chat of a simplified process for preparing the gluconate-containing support medium provided herein, showing the preparation steps of four exemplary embodiments thereof.

As discussed herein, the calcium-alginate hydrogel is formed with the intention to slow the gelation process, which can be done in several ways, one of which includes the use of water-insoluble calcium salts in the presence of an acidifier. In some embodiments, the water-insoluble calcium salt is calcium carbonate, and the acidifier is glucono-S-lactone.

This hybrid hydrogel, formed under these conditions (slow gelation in the presence of the other soluble polymer), is thereafter pulverized (crushed and grinded) into fine particles which is more amenable to afford small uniformly sized particles.

These particles are thereafter washed by suspending the particles in a solution that maintains the concentration of calcium in the wash solution (using, e.g., highly soluble CaCl₂)), thereby preventing dissociation of the crosslinked hydrogel. The suspended particles are thereafter drained form the wash solution, and can be re-suspended for the next steps. During the wash step, most or essentially all the remaining non-bound substances, such as loose soluble polymer chains, the low-solubility calcium salt, the acidifier, unbound calcium ions and non-crosslinked alginate strands, are removed from the particles and the slurry carrier (the solution which the particles are suspended in).

The term “washed”, as used herein in the context of a mid-process hydrogel and/or the finished product, refers to both the process step and the resulting hydrogel after effecting the step. Since a hydrogel in its hydrated (gel-like) state is a porous network of crosslinked and entangled polymer strands in an aqueous medium, a hydrogel typically allows the passage and exchange of aqueous solutions through its structural matrix. Washing may be effected as follows: once the structural matrix is formed and a hydrogel is afforded in the original dispersion medium, one may soak the hydrogel in a medium (e.g., aqueous solution) that is different than the original dispersion medium, with or without first draining the hydrogel from excess original dispersion medium (e.g., by straining and/or centrifugation), thereby replacing the post-wash medium in the hydrogel with a wash medium. A washed hydrogel is therefore a hydrogel which has undergone replacement of its aqueous medium, whereas the concentration of the soluble ingredients in the washed hydrogel correspond to their concentration in wash medium. The exception to this definition of a washed hydrogel is the concentration of the non-alginate soluble polymer, which is said to be present in the hybrid hydrogel either as an integral part of the polymeric network as well as a solute in the dispersion medium, and in the dispersion medium at least at trace amounts even after washing the hybrid hydrogel with a wash solution that does not include the non-alginate soluble polymer.

One of the rudimentary concepts underlying of the present invention is the concept of a hybrid hydrogel, which is the formation of a hydrogel with more than one type of polymer, one that is being crosslinked into the basic polymeric network of the hydrogel, and another that remains soluble under the crosslinking conditions, namely does not undergo crosslinking, however, it is an integral part of the network as a plurality of caged and/or entangled strands, e.g., non-covalent bonding between two polysaccharides.

A hybrid hydrogel, according to embodiments of the present invention, is a molecular megastructure that is afforded by the interactions between two polysaccharides within a hydrogel, wherein a first polysaccharide is alginate and the second polysaccharide is the non-alginate soluble polymer. The first polysaccharide undergoes crosslinking with calcium to form the network, while the second polysaccharide is incorporated into the network through various bonding mechanisms, including direct and/or water-mediated hydrogen bonds (H-bonds), mechanical entanglement, and caging. The formation of the hydrogel involves crosslinking the first polysaccharide chains to create a polymer network. This is typically achieved through crosslinking agents, such as calcium ions. The second polysaccharide, is introduced into this network during or after its formation. The incorporation of the second polysaccharide occurs through multiple bonding mechanisms such as H-bonds, mechanical entanglement, and the caging effect. These interactions between the crosslinked and incorporated polysaccharides significantly influence the properties of the hydrogel as a support medium in the form of a plurality of hybrid hydrogel particles.

The term “wash”, in the context of processing the support medium provided herein, may also encompass the term “treat”, since both terms refer to a step wherein the particulate hydrogel is contacted with an aqueous solution containing any given solutes therein. For example, when stating that a particulate hydrogel is treated with a substance, it is equivalent to a statement that particulate hydrogel is washed with a medium containing the substance; it is also equivalent to a statement that one dispersion medium is replaced with another dispersion medium.

The washed hybrid hydrogel particles are thereafter treated with gluconate, now added as a solution of a non-calcium gluconate salt (e.g., sodium gluconate). The treatment is the addition of the gluconate solution to the drained pellet of the particles (e.g., a wash medium comprising non-calcium gluconate salt), or to a non-drained suspension of the washed particles, whereas the concentration of the gluconate in the final pellet or suspension is at least 2-5 mM. It is noted herein that even if some gluconate, originating from GDL that was used as an acidifier in the initial hydrogel formation step, remains in the hydrogel, its concentration would be negligible due to washing, and its contribution to the final concentration of gluconate in the support medium is regarded as negligible. According to some embodiments of the present invention, these hybrid hydrogel particles containing gluconate, are ready for use as the support media provided herein; the non-drained washed particles treated with non-calcium gluconate salt are represented by “support medium; embodiment 1” in FIG. 1.

Optionally, after the treatment with non-calcium gluconate salt solution the hybrid hydrogel particles are treated (contacted; washed) with a solution containing the non-alginate soluble polymer in order to set the final concentration of the non-alginate soluble polymer in the hybrid hydrogel at the range of 0.0001-4% w/w or % w/v of the total weight or volume of the hydrogel. In some embodiments, the concentration of the non-alginate soluble polymer in the hybrid hydrogel may be increased to more than 4% w/w or % w/v of the total weight or volume of the hydrogel, namely up to or at least 5% w/w or % w/v, at least 6% w/w or % w/v, at least 7% w/w or % w/v, at least 8% w/w or % w/v, at least 9% w/w or % w/v, at least 10% w/w or % w/v, at least 11% w/w or % w/v, at least 12% w/w or % w/v, at least 13% w/w or % w/v, at least 14% w/w or % w/v, or at least 15% w/w or % w/v.

An embodiment that represent such particles that are treated with non-calcium gluconate salt and a non-alginate soluble polymer are represented by “support medium; embodiment 2” in FIG. 1.

Optionally, after the treatment with non-calcium gluconate salt solution the hybrid hydrogel particles are drained from the non-calcium gluconate salt solution in order to yield a compact pellet, and the drained particles treated with non-calcium gluconate salt are represented by “support medium; embodiment 4” in FIG. 1.

Further optionally, after the treatment with non-calcium gluconate salt solution the hybrid hydrogel particles are drained from the non-calcium gluconate salt solution and are treated (contacted) with a solution containing the water-soluble polymer in order to set the final concentration of the soluble polymer in the hybrid hydrogel at the range of 0.0001-4% w/w or % w/v of the total weight or volume of the hydrogel. These particles treated with non-calcium gluconate salt and the soluble polymer are represented by “support medium; embodiment 3” in FIG. 1.

The resulting support medium is afforded typically as a slurry of fine, homogeneous plurality of hydrated hybrid hydrogel particles, that include detectable amounts of gluconate and detectable or trace amounts of the non-alginate soluble polymer.

In some embodiments, the resulting support medium can be dried to a fine powder that can be rehydrated with an aqueous medium that includes non-calcium gluconate salt and other optional ingredients.

According to some embodiments, the non-alginate soluble polymer is xanthan gum.

### Hybrid-Hydrogel-Based Particulate Support Media:

The support medium for 3D-printing provided herein is a plurality of hydrogel particles afforded by forming a calcium-alginate hybrid hydrogel under specific conditions and pulverizing the same into the plurality of particles. In general, the hybrid hydrogel particles are:
composed of a network of alginate chains that are cross-linked by calcium ions, and further incorporating a non-alginate soluble polymer in the network;
small, typically in the range of 0.1-50 μm in diameter;
exhibit a size distribution of less than 50% RSD;
porous, with a high surface area to volume ratio;
soaked and surrounded by a medium, referred to herein a dispersion medium.

The support medium also includes a dispersion medium present within and surrounding the hydrogel particles, which plays an important role in determining the properties of the final support medium. The dispersion medium is present at least within the particles, and may also be present between particles at a minimal amount or in excess. Once the hydrogel is formed, the dispersion medium can be augmented and replaced, and its amount may be changed, as long as the hydrogel is maintained, namely, as long as the network of crosslinked polymeric chains is immersed in the dispersion medium, namely that the hydrogel is hydrated with the dispersion medium.

In terms of amount, the dispersion medium is part of the hydrogel, as stated in the definition of a hydrogel, and in practice, the support medium provided herein includes at least the amount of a dispersion medium within the polymeric network, and may include excess dispersion medium. The volume ratio of particles to excess dispersion medium, defined as the dispersion medium that is in between the particles, also has an effect on the performance of the support medium, and can be set by the user to accommodate various purposes and needs on the print-job. When using the support medium provided herein for 3D-printing, the hybrid hydrogel particles can be more tightly packed or less, depending on the volume ratio of particles to excess dispersion medium, wherein a support medium characterized by a minimal amount of dispersion medium may be achieved by draining the hybrid hydrogel particles from excess dispersion medium prior to use. In such embodiments, the hydrogel particles still fall under the definition of a hydrogel, namely that the hydrogel particles include by definition a dispersion medium; in the context of some embodiments of the present invention, drained hydrogel particles are those that no dispersion medium can further be removed by mild means, such as straining and/or centrifugation, whereas excess dispersion medium can be removed from the hydrogel particles without collapsing the hydrogel.

The term “drained” in the context of a plurality hybrid hydrogel particles refers to a plurality of hydrogel particles which have been separated from excess dispersion medium by straining and/or centrifugation, leaving the hydrogel particles hydrated with the dispersion medium.

According to some embodiments of the present invention, the support medium provided herein can be used as a drained plurality of hybrid hydrogel particles, i.e., in a drained form of the plurality of hybrid hydrogel particles.

According to some embodiments of the present invention, the support medium provided herein can be used as a slurry, wherein the volume ratio of drained hybrid hydrogel particles to excess dispersion medium ranges from 1:0.001 to 1:3. In other words, the support medium provided herein can be used as a concentrated slurry having 1 part drained particles and 0.001 parts excess dispersion medium, as well as a dilute slurry having 1 part drained particles and up-to 3 parts excess dispersion medium, and any intermediate ratio therebetween.

It is noted herein that since the hybrid hydrogel is formed while a non-alginate soluble polymer is present during the gelation, it is assumed that some of the non-alginate soluble polymer becomes entangled or otherwise incorporated/entrapped in the polymeric network of the hydrogel. It is also assumed that some of the non-alginate soluble polymer remains unbound to the polymeric network, not being entrapped inside the network, and soluble in the dispersion medium. The amount of the network-bound/entrapped portion of the non-alginate is assumed to be substantially invariable under washes, at least in trace amount. The amount of the unbound portion of the non-alginate soluble polymer can be controlled by setting its concentration in the dispersion medium.

Thus, according to an aspect of embodiments of the present invention, there is provided a plurality of hybrid hydrogel particles, suitable for use as support medium in 3D-printing, wherein the hybrid hydrogel includes calcium-alginate, a non-alginate soluble polymer, and a non-calcium salt of gluconate.

The support medium provided herein is transparent, heat-stable, safe and (bio)degradable hydrogel-based particulate support medium for 3D printing, wherein the plurality of hybrid hydrogel particles is characterized by an average size that ranges from 0.1 μm to 50 μm, and a size distribution of less than 50% RSD. It is the chemical composition of the hybrid hydrogel particles contributes to the ability of the pulverization step to afford the desired properties of small size and narrow size distribution of the particles, as well as to contribute to the transparency of the product.

In some embodiments, the hybrid hydrogel particles having an average size that ranges 0.1-5 μm, or 0.1-10 μm, or 0.1-15 μm, or 0.1-20 μm, or 0.1-25 μm, or 0.1-30 μm, or 0.1-35 μm, or 0.1-40 μm, or 0.1-45 μm, or 0.1-50 μm. In some embodiments, the particles are sieved though a filter with an upper size cutoff selected at 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, or 30 μm, making the upper size limit set by the filter's cutoff.

The plurality of calcium alginate hydrogel particles, according to embodiments of the present invention, are characterized by being essentially discrete (low level of aggregation) and having a relatively narrow size distribution, or a lower coefficient of variation (CV). CV is also known as relative standard deviation (RSD), which is a standardized measure of dispersion of a probability distribution or frequency distribution, and is often expressed in percent. RSD is defined as the ratio of the standard deviation (a) to the mean (p), or RSD=(Standard Deviation/Mean)×100%. According to some embodiments of the present invention, the plurality of calcium alginate hydrogel particles is characterized by a particle size distribution of less than 50%, less than 40%, less than 30%, less than 20%, less than 10%, or less than 5% RSD.

The support medium comprises calcium-alginate hydrogel crosslinked network, a non-alginate soluble polymer as a trace ingredient (below detectable levels in some substance detection methods) or in a detectible amount of, and gluconate anion (as a non-calcium salt thereof). There may be minute/trace amounts of other substances in the hydrogel, which persist even after repetitive washes, however, these trace amounts are either non-detectable or seem not to affect the properties of the final product. Other substances, such as low millimolar concentration of calcium (to support the integrity of the hydrogel and to prevent its disintegration) and physiological concentration of salts and nutrients (to support cell viability) may also be present in the final product. These substances may be introduced to the support medium during or after the washing process, and can be set and defined by the user/producer to match a specific application.

The amount of gluconate in the dispersion medium is measurably controlled, and detectable by standard chemical analysis tools. It is set by washing/treating the hybrid hydrogel particles with a solution comprising non-calcium gluconate salt at any given concentration, e.g., at least 2-5 mM. Thus, according to some embodiments of the present invention, the dispersion medium of the hybrid hydrogel particles of the support medium provide herein, comprises non-calcium gluconate salt at a concentration of at least 2 mM, 3 mM, 4 mM, 5 mM, 6 mM, 7 mM, 8 mM, 9 mM or at least 10 mM. Preferably, the gluconate is added to the particles as sodium D-gluconate solution.

As the calcium ions crosslink with the alginate polymer chains, they become incorporated into the structure of the hydrogel. In a fully formed and hydrated calcium-alginate hydrogel, the concentration of calcium ions is typically determined by the initial concentration of calcium ions in the solution used for gelation and the degree of crosslinking that occurred during the gelation process, and whether the formed hydrogel had been washed, how many times it had been washed, and with which wash solution (i.e., a wash solution may include calcium ions or calcium sequestrant). The actual concentration of calcium ions within the hydrogel can be measured experimentally using techniques such as atomic absorption spectroscopy or inductively coupled plasma mass spectrometry (ICP-MS). The concentration will depend on factors such as the initial concentration of calcium in the gelation solution, the ratio of calcium ions to alginate molecules, the type of alginate and the extent of crosslinking that occurred during gelation.

In the context of some embodiments of the present invention, the concentration of calcium in a fully formed and hydrated calcium-alginate hydrogel can vary depending on the concentration and type of alginate and the concentration calcium ions used to form the hydrogel. In general, the concentration of calcium is typically in the range of 3-30 mM. However, it is possible to achieve higher or lower concentrations depending on the desired properties of the hydrogel. For example, a calcium-alginate hydrogel with a calcium concentration of 10 mM has a higher mechanical strength than a hydrogel with a calcium concentration of 3 mM; the higher calcium concentration may render the hydrogel more brittle.

As discussed hereinabove, the amount of the non-alginate soluble polymer that is incorporated into the polymeric network of the hydrogel is taken as essentially invariable compared to the amount thereof in the dispersion medium that is present in and surrounding the particles. It is noted that in some embodiments, the hybrid hydrogel is formed with a predetermined amount of the non-alginate soluble polymer, and thereafter the hybrid hydrogel is washed with a solution that removed unbound ingredients therefrom; in such embodiments wherein the wash solution does not contain the non-alginate soluble polymer or contains a low concentration thereof, it is possible that the particles of the hybrid hydrogel incorporate more of the non-alginate soluble polymer that can be detected in the dispersion medium; this can be verified by subjecting the hybrid hydrogel to analytical methods as presented herein, some of which are destructive (e.g., monosaccharide analysis following polysaccharide isolation and depolymerization, and enzymatic digestion fingerprinting). Hence, when referring herein to the amount/concentration of the non-alginate soluble polymer in the support medium provided herein, in some embodiments it refers to the amount/concentration of the non-alginate soluble polymer in the dispersion medium that fills the porous network of the particles as well as the space between the particles, while assuming that the non-alginate soluble polymer that is incorporated into the polymeric network contributes somewhat mildly to its concentration in the dispersion medium, and therefore the minimal amount of the non-alginate soluble polymer in the support medium is said to be as low as about 0.0001% w/w or % w/v of the total weight or volume of the support medium.

The maximal amount/concentration of the non-alginate soluble polymer in the dispersion medium of the support medium that is not incorporated into the polymeric network, is limited by the solubility thereof in the dispersion medium, and can be set by treating/washing the plurality of hybrid hydrogel particles. In some embodiments, the concentration of the non-alginate soluble polymer in the support medium ranges 0.0001-4% w/w or % w/v of the total weight or volume of the support medium. In some embodiments, the maximal concentration of the non-alginate soluble polymer may be increased to more than 4% w/w or % w/v of the total weight or volume of the hydrogel, namely up to or at least 5% w/w or % w/v, at least 6% w/w or % w/v, at least 7% w/w or % w/v, at least 8% w/w or % w/v, at least 9% w/w or % w/v, at least 10% w/w or % w/v, at least 11% w/w or % w/v, at least 12% w/w or % w/v, at least 13% w/w or % w/v, at least 14% w/w or % w/v, or at least 15% w/w or % w/v.

Exemplary non-alginate soluble polymers that may be used in the context of a hybrid hydrogel as described and provided herein, include, without limitation, a natural or synthetic hydrocolloid, a polysaccharide hydrocolloid, xanthan gum, gellan gum, guar gum, konjac gum, gum Arabic (acacia), locust bean gum, starch, methyl cellulose, hydroxyethyl cellulose, carboxymethyl cellulose, pectin, carrageenan and agarose, and any combination thereof.

It is noted that the diffusion limitations of the non-alginate soluble polymer in and out of the hybrid hydrogel particles should be considered. The solute concentration in the dispersion medium between the particles can be easily controlled by the composition of the wash medium. However, the solute's diffusion capacity and its movement within the calcium-alginate network can influence the medium's content inside the particle. For relatively large non-alginate soluble polymers such as xanthan gum, this could potentially affect its concentrations within the particles.

According to some embodiments of the present invention, the hybrid hydrogel particles include a non-alginate soluble polymer at a concentration that ranges 0.0001-4% w/w or % w/v of the total weight or volume of the support medium. As for the exact amount of the non-alginate soluble polymer that is incorporated into the polymeric calcium-alginate crosslinked network, it may be determined by more advanced methodologies, as described hereinbelow.

According to some embodiments of the present invention, the soluble polymer is xanthan gum. In case of xanthan gum having a 2M Da (molecular weight) chain, 0.0001-4% is equivalent to about 0.0005-20 micromolar (1%=5 micromolar).

The concentration of xanthan gum at trace amounts in the hydrogel particles may be evaluated by calculation using its original concentration in the dispersion medium, the contents, volume and number of the wash solution(s), while considering the amount of network-bound xanthan gum as substantially invariable (e.g., non-leachable). The detection of xanthan gum in trace amounts, and the amount of network-bound xanthan gum may also be carried out using NMR spectroscopy, FTIR, monosaccharide analysis following polysaccharide isolation and depolymerization, enzymatic fingerprinting, as well as xanthan gum-specific antibodies. An indirect method for assessing the concentration of xanthan gum in the slurry of hydrogel particles may even be carried out using polymerase chain reaction (PCR) with primers that are specific for the ribosomal DNA of X. campestris, the bacterial source of xanthan gum.

The final product can be in the form of a drained pellet of particles that can be re-suspended in an aqueous solution, or in the form of a slurry (particles suspended in an aqueous solution). According to some embodiments of the present invention, the hydrogel-based particulate support medium provided herein is generally a plurality of hydrogel particles hydrated by an aqueous solution in the form of a slurry or a suspension. The amount of the aqueous solution may vary from essentially no excess thereof (a drained slurry) up to a diluted suspension of the particles at a ratio that allows the printed object to be supported and formed adequately. The preferable amount of dilution is determined within the routine experimental procedures of a person skilled in the art.

In some embodiments, the support medium provided herein may be dehydrated and provided as a dry rehydratable powder. Typically, the rehydration medium includes gluconate and other optional ingredients.

“Suspension”, “slurry”, and “powder” are terms used to describe different states of the support medium provided herein, with varying amounts of a liquid aqueous medium. The main differences between these stated lie in the relative amount of aqueous medium and the consistency or flowability of the resulting mixture. A powder typically contains very little liquid relative to the solid particles, and consists mostly of solid particles that are dry and fine, often in the form of small, individual particles or grains. A slurry contains a moderate amount of liquid compared to the solid particles, and it is typically a thick, viscous mixture where solid particles are suspended in a liquid, and the mixture has a somewhat uniform consistency; a drained plurality of hydrogel particles is typically referred to as a slurry. A suspension contains a relatively large amount of liquid compared to the solid particles, and it is a heterogeneous mixture where solid particles are dispersed in a liquid medium but do not dissolve or substantially settle, or require agitation to keep the solid particles evenly distributed if they tend to settle over time due to gravity.

According to some embodiments, the support medium provided herein is sterile and free of any toxins or other factors that may adversely affect an article of manufacturing being formed therewithin.

Moreover, as the fine and homogeneous particles of the support medium provided herein, can easily flow throw very fine needles, and can therefore also serve as a sacrificial material in 3D printing of hollow structures.

The high see-through transparency of the support medium to visible light, according to some embodiments of the present invention, allows the printing process to be monitored at real time: the user can inspect the printed construct while fabricated and modify printing parameters accordingly without interrupting the procedure. The high see-through transparency of the media may also allow automatic calibration and monitoring of the printing process by 3D printers equipped with compatible optical sensors. This enables the user to calibrate and tune the printer conveniently and accurately, what results in a refined printout. Finally, the transparency of the support media may enable curing of light-curable printed materials by external illumination. Thus, according to some embodiments of the present invention, the plurality of calcium alginate hydrogel particles is substantially transparent to visible light.

### A Selling Unit of the Support Medium:

According to an aspect of some embodiments of the present invention, there is provided a selling unit, comprising the support medium provided herein. The selling unit may optionally be in a form of a sealed and oxygen-proof container that contains an amount of the support medium suitable for a specific printing machine or a specific printing bath size.

According to some embodiments, the selling unit contains a support medium in the form of drained plurality of hybrid hydrogel particles, which is a hydrated form of the hydrogel at or near the minimal amount of dispersion medium. In such embodiments the user may be instructed to add a dilution solution to the drained plurality of hybrid hydrogel particles in order to arrive at an adequate density of the support medium.

According to some embodiments, the selling unit contains a support medium in the form of slurry containing a predetermined amount of dispersion medium. The volume ratio between the particles and the excess dispersion medium ranges 1:0.001 to 1:3.

According to some embodiments, the selling unit contains dehydrated particles of the support medium, wherein the user is instructed to add an aqueous solution thereto in order to rehydrate the hydrogel and arrive at an adequate hydration and density of the support medium.

According to some embodiments of the present invention, the selling unit includes a packaging material, and identified in print, or on said packaging material, for use as a support medium in a 3D printing machine and process. In some embodiments, the selling unit includes separate container containing an aqueous solution intended for rehydration and/or density adjustment of the support medium.

It is expected that during the life of a patent maturing from this application many relevant hydrogel-based particulate support media will be developed and the scope of the term hydrogel-based particulate support media is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Gluconate Effect

The support medium provided herein was prepared essentially as described in WO 2019/234738, with specific modification of adding gluconate to the pre-formed, pre-pulverized and washed hydrogel. This addition of gluconate to the hydrogel after it has formed, turned into particles and washed is not equivalent to the use of glucono-δ-lactone as an acidifier to assist in calcium ion solubilization from calcium carbonate during the gelation step of the process.

The support medium provided herein can be prepared, according to some embodiments of the invention, by treating pre-formed calcium-alginate hydrogel with gluconate solution after pulverizing and washing the hydrogel from excess calcium and other non-bound substances, and optionally before addition of the soluble polymer (e.g., xanthan gum). As will be shown below, the mason for this order of preparation is to allow the user the option to discard the extra volume of solution added to the particulate pellet after the addition of sodium gluconate solution. This draining step is performed by centrifugation of the particulate pellet after vigorous vortexing and an incubation period (for example 15 min), during which gluconate diffuses into the hydrogel. After the soluble polymer (e.g., xanthan gum) is added to the hydrogel, the particulate pellet cannot be centrifuged to get a more compact pellet, as precipitation of calcium-alginate particles will not be efficient.

Briefly, a hybrid hydrogel, composed of 0.25-0.5% (w/v) sodium alginate (tested with Protanal® LF 10/60 FT and Protanal® LF200 FTS, FMC BioPolymer, or Kimica Algin I-3G by KIMICA corporation), 0.125-0.5% (w/v) xanthan gum (tested with Xantural 180, CP Kelco, Sigma G1253, xanthan gum from Xanthomonas campestris), 7.5-15 mM calcium carbonate, and 15-30 mM D-(+)-gluconic acid 8-lactone (GDL as acidifier and a weak calcium sequestrant) was made by mixing the following ingredients:
25-50% v/v of 1% (w/v) sodium-alginate solution (0.22 μm filtered);
12.5-25% v/v (or centrifuged pellet) of 60 mM suspension of calcium carbonate (CaCO₃) in deionized water, sonicated to reduce the size of the calcium-carbonate particles (autoclaved);
0-50% v/v of deionized water (autoclaved or filtered @0.22 μm); and
12.5-50% v/v of 1% (w/v) xanthan gum in 150 mM NaCl solution (autoclaved; can also be added as separate solutions of xanthan gum and NaCl).

It is noted that a higher percentage of xanthan may be required if using an alginate batch that form stronger gels. Alginate testing can predict such situation as a routine step in the process, supporting adjustment of some process parameters.

The above ingredients, sodium-alginate, calcium carbonate, xanthan gum, NaCl and water, were mixed for at least 30 minutes, and thereafter a fresh GDL solution (0.22 μm filtered) was add to a final concentration of 15-30 mM.

An embodiment of a specific formulation, preferred in terms of printout support capacity and see-through optical clarity is given below:
42.5% (v/v) of 0.75% (w/v) sodium alginate solution (Protanal LF200 FTS, FMC Biopolymer); 22.5% (v/v) of 1.111% (w/v) xanthan gum solution (Xantural 180, CP Kelco); 2.5% (v/v) of 150 mM NaCl solution, 15.94% (v/v) of 60 mM suspension of calcium carbonate, 16.56% (v/v) deionized water and 19.15 mM GDL.

The mixture was vigorously stirred for 3 minutes, after which the stirring speed was lowered to generate a fine turbulence that bends the surface of the liquid. When the bend was no longer visible due to the increase in the viscosity of the reaction mixture, the stirring was stopped and the mixture was incubated uninterrupted, at room temperature, for 24 hours.

The resulting hydrogel was manually broken into chunks to which sterile deionized water at a volume of 4 times the volume of the hydrogel was added to a total volume of 5 times the volume of the hydrogel. The water-suspended hydrogel chunks were thereafter homogenized into fine particles using an electric homogenizer, and the homogenate was incubated over-night at 4 °C to allow dissolution of remaining traces of CaCO₃.

In order to remove excess unbound substances, including GDL, the homogenized hydrogel suspension was centrifuged at 15,777 G for 20 minutes at 4 °C. Thereafter the pellet was washed 2 times with sterile HEPES-buffered saline (pH 7.4) containing 1.5 mM CaCl₂), added at a volume equal to the discarded supernatant, by repeated centrifugation at 15,777 G for 20 minutes at 4 °C and re-suspension by vigorous mixing. Excess calcium ions, GDL and loose polymer strands are washed away during these washing steps, leaving transparent granular media composed of the hybrid hydrogel particles in the form of a pudding-like pellet.

To quantify the degree of clarity (transparency, translucency, lucidity), and to demonstrate the uniqueness of post-washing addition of gluconate, the present inventors measured the clarity of the support medium after treatment with either a control additive (HEPES buffered saline with 1.5 mM CaCl₂), used as a washing buffer in the support medium preparation process), phosphate buffered saline (PBS; a biocompatible, non-cytotoxic formula containing phosphate that is also known for its calcium-sequestration ability), and sodium D-gluconate. These experiments also compare the presently disclosed invention to the provisions disclosed in WO 2019/234738.

Specifically, in the process of producing the support medium, after washing with HEPES buffered saline (pH 7.4) that contains 1.5 mM CaCl₂), the drained slurry was supplemented with 5:1 (w/w or v/v, slurry-to-supplement) of the following supplement solutions:
1) HEPES buffered saline (pH 7.4) with 1.5 mM CaCl₂) (denoted by “HEPES B.S.”);
2) Dulbecco's Phosphate Buffered Saline (DPBS), which is provided devoid of calcium and/or magnesium (denoted by “PBS”); and
3) 150 mM sodium D-gluconate (denoted by “D-Gluc”).

The different particulate slurries were vortexed vigorously and incubated at room temperature for 30 minutes. Thereafter the particulate slurries were centrifuged and drained, and xanthan gum solution (1% w/v) was added to the slurry at a 1:20 volume ratio (reaching 0.05% w/v xanthan concentration in the particulate slurries). The particulate slurries were thereafter vortexed vigorously and centrifuged at low speed to extract air bubbles.

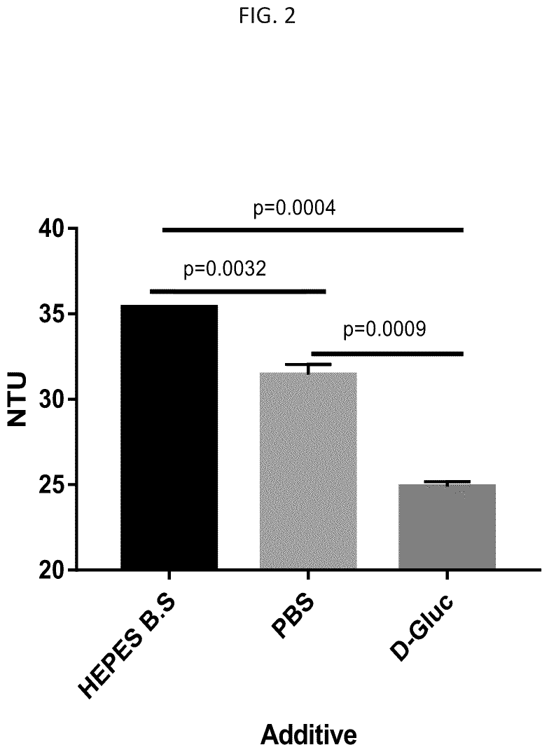

The turbidity of the mixtures was analyzed using LaMotte 2020i turbidimeter, with HEPES buffered saline (pH 7.4) with 1.5 mM CaCl₂) serving as a blank solution, and the results are presented in FIG. 2.

FIG. 2. presents the results of a comparative turbidity test conducted for the support medium following treatment with each of the abovementioned supplements, whereas the support media samples were supplemented with 5:1 (w/w or v/v) of the following solutions: HEPES buffered saline (pH 7.4) with 1.5 mM CaCl₂ (“HEPES B.S”); Dulbecco's Phosphate Buffered Saline, devoid of calcium and magnesium (“PBS”); 150 mM sodium D-gluconate (“D-Gluc”), and the results are given in Nephelometric Turbidity Units (NTU).

As can be seen in FIG. 2, treatment with gluconate significantly reduced the turbidity of the support media from 35.4 Nephelometric Turbidity Units (NTU) in the “HEPES B.S” control to 24.9 NTU. It has also been found that gluconate treatment is superior to PBS, with the latter reducing the turbidity of the support media to only 31.43 NTU.

### Example 2

### Support medium with improved clarity

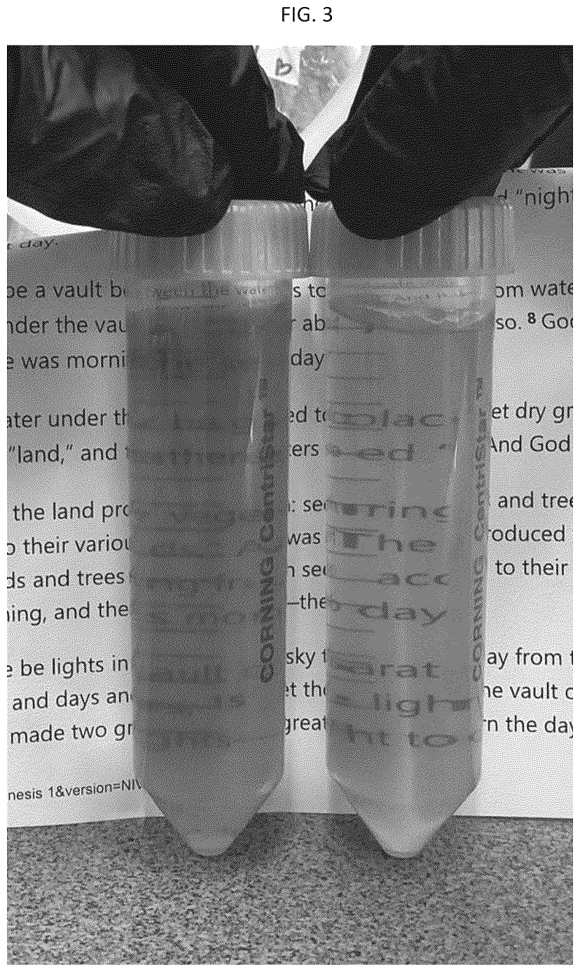

A visual demonstration of the effect of the treatment with sodium D-gluconate is demonstrated in FIG. 3. In this demonstration, during the production process of the support medium, after washing with cell culture medium DMEM/F-12 (Dulbecco's Modified Eagle Medium/Nutrient Mixture F-12), the drained slurry was supplemented with 5:1 (w/w or v/v) of DMEM/F-12 as an untreated control, or 150 mM sodium D-gluconate. The different mixtures were vortexed vigorously and xanthan gum solution (1% w/v) was added to the slurry at a 1:20 volume ratio (reaching 0.05% w/v concentration.) The mixtures were then vortexed vigorously and centrifuged at low speed to extract air bubbles.

FIG. 3 is a black and white photograph that compares the transparency of the support medium following treatment with DMEM/F-12 cell culture medium (left) or 150 mM sodium D-gluconate (right).

As can be seen in FIG. 3, the improvement in transparency is clearly evident by observing the two 50 ml Falcon tubes (internal diameter of 2.7 cm) filled with support medium and juxtaposed.

### Example 3

### Cell Response to Gluconate

The cytocompatibility of gluconate is an important prerequisite for biofabrication applications in which living cells are one of the components of the printout. In addition, as been demonstrated by the present inventors, other potent chelators, such as citrate, EDTA, EGTA, and polyphosphates, which bind calcium ions with high affinity, resulted in a rapid destabilization of the hydrogel and the loss of its supporting capability. Moreover, these calcium-chelating agents have the potential to adversely affect cells through a direct cytotoxic activity or by depravation of calcium that is essential for cell metabolism and activity.

To demonstrate the benign effect of sodium D-gluconate at the relevant concentration on live cells, human endothelial cells were seeded (1×10⁵ cells/well in 24 well plate) in growth media supplemented with 5:1 (V/V, growth media-to-supplement) ratio PBS (a positive control of a known non-toxic material), 5:1 V/V ratio 150 mM sodium D-gluconate solution, and 333:1 V/V ratio ProClin™ 915 (a negative control of a known toxic material).

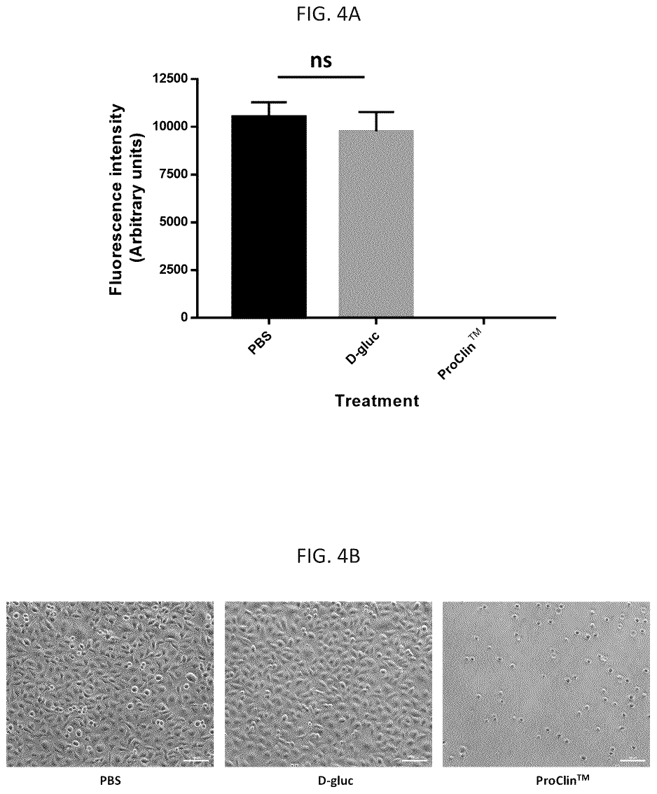

FIG. 4A-B present the result of the cell viability assays, as determined 48 hours post-seeding, wherein FIG. 4A shows fluorescence as a function of cell growth (quantified using PrestoBlue™ reagent) and FIG. 4B shows microscopic images of cells.

As can be seen in FIGS. 4A-B, cell growth and morphology in the presence of sodium D-gluconate is comparable to treatment with PBS, indicating on the cytocompatibility of D-gluconate.

### Example 4

### Printing Quality

In order to demonstrate that the sodium D-Gluconate does not interfere with the capacity of the alginate-xanthan hybrid hydrogel particulate media to support the printout, grid-shaped structures were 3D printed through a 30 G needle inside support media treated with saline as a control (5:1 V/V ratio saline) or with sodium D-gluconate (5:1 V/V ratio 150 mM sodium D-gluconate solution).

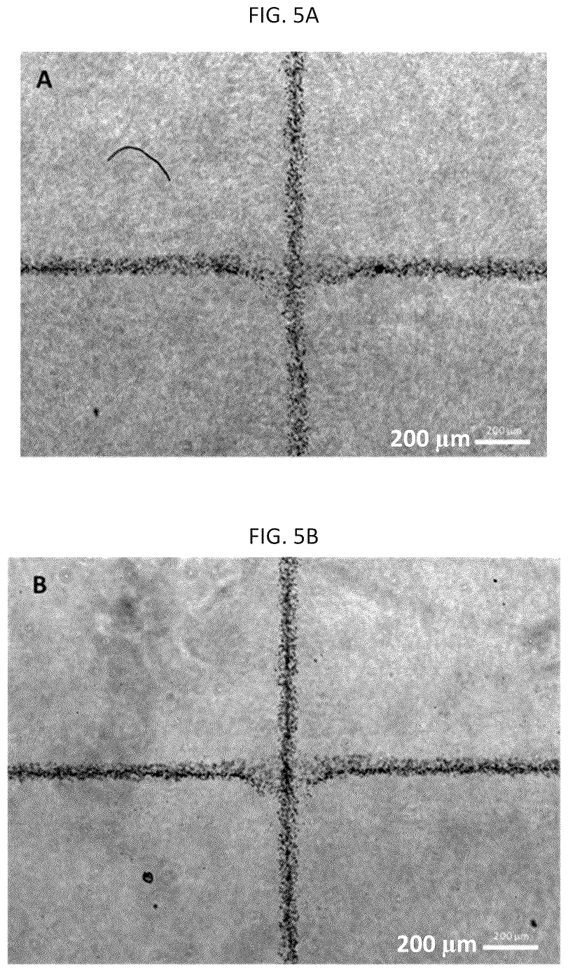

FIGS. 5A-B present light-microscopy images, taken with 4× objective lens (scale bars=200 micrometer), showing the effect of sodium D-gluconate on printing quality, whereas FIG. 5A is a pattern printed as a control experiment using 5:1 V/V ratio saline, and FIG. 5B is the same pattern printed using 5:1 V/V ratio 150 mM sodium D-gluconate solution.

As can be seen in FIGS. 5A-B, the deposited strands were adequately supported in both media, maintaining their straight geometry and smooth contour, proving that gluconate has no adverse effect on 3D printing quality.

### Example 5

### Pre- Vs Post-Gelation Addition of Gluconate

Calcium gluconate (not to be confused with sodium gluconate) is known in the art as a calcium source for generation of calcium-alginate hydrogels, in which the gluconate ion acts as a calcium sequestrant that compete with the alginate on calcium binding during the process of gelation. While reducing the present invention to practice, the present inventors studied the difference of calcium gluconate versus sodium gluconate on the formation of the hybrid hydrogel, and have shown that sodium gluconate, and other non-calcium salts of gluconate, exert optimal effect on the particulate support medium when used on already prepared, washed hybrid hydrogel.

The following experimental proof of concept was conducted in order to show that treatment with sodium D-gluconate is superior when applied to calcium-alginate post-gelation (e.g., treating the support medium subsequent to the washing steps with 5:1 V/V ratio 150 mM sodium D-gluconate solution, “D-Gluc treatment”), as opposed to having the same amount of sodium D-gluconate (25 mM) during the initial gel preparation (“D-Gluc in gel prep+Saline treatment”).

In addition, the effect of treating support media with phosphate, another delicate, biocompatible chelator, (PBS, 5:1 V/V ratio, “PBS treatment”), was also studied. An “untreated” (5:1 V/V ratio, saline) support medium was used a control reference (“Saline treatment”). The turbidity was measured using Lovibond Photometer System MD600.

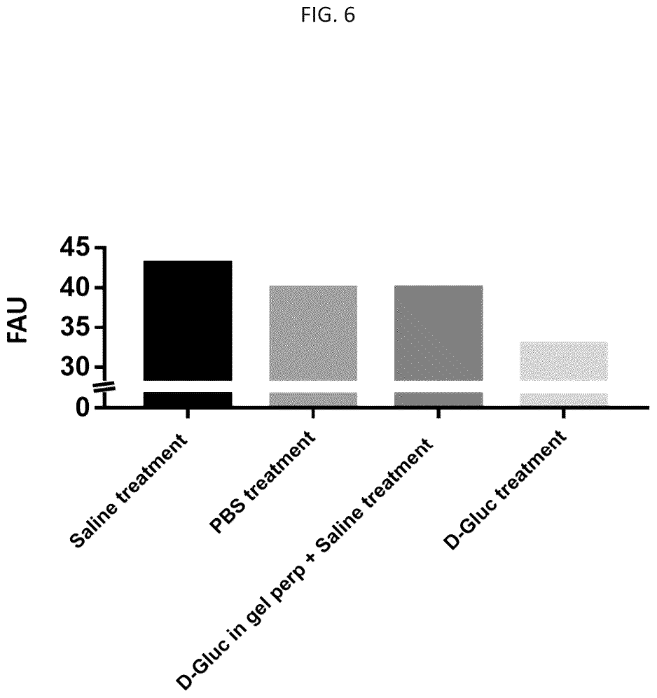

FIG. 6 presents a comparative chart, showing the turbidity values measured for a gel treated with no gluconate (“Saline treatment”), with another biocompatible calcium chelator (“PBS treatment”), with sodium gluconate added during the initial gel preparation and wetted with saline (“D-Gluc in gel prep+Saline treatment”), and with a gel that was treated with sodium D-gluconate solution after preparation (“D-Gluc treatment”).

As can be seen in FIG. 6, post-gelation treatment with sodium D-gluconate solution resulted in a superior reduction in the turbidity of the support, compared to support prepared with D-gluconate present in the initial gelation process. This experiment supports the inventors' contention that while using calcium sequestrants in the gelation process has been suggested in the art and implemented also in WO 2019/234738, it is the treatment of pre-formed, washed calcium-alginate hydrogel with non-calcium salt of D-gluconate that further improved the optical qualities of the hydrogel.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A 3D-printing support medium, comprising:
a plurality of hybrid hydrogel particles, and a dispersion medium, said hybrid hydrogel comprises calcium-alginate and a non-alginate soluble polymer, wherein said dispersion medium comprises at least 2 mM gluconate, wherein said gluconate is an anion of a non-calcium gluconate salt.

2. The support medium of claim 1, wherein said particles are characterized by an average size that ranges from 0.1 μm to 50 μm, and a homogeneity characterized by a particle size distribution of less than 50% RSD.

3. The support medium of claim 2, characterized by being substantially transparent to visible light.

4. (canceled)

5. The support medium of claim 1, wherein said non-calcium gluconate salt is sodium gluconate, potassium gluconate, zinc gluconate, magnesium gluconate, and any combination thereof.

6. The support medium of claim 1, wherein said non-alginate soluble polymer is xanthan gum.

7. The support medium of claim 6, comprising xanthan gum at a concentration that ranges 0.0001-4% w/w or % w/v of the total weight or volume of the support medium.

8. The support medium of claim 1, in a drained form of said plurality of hybrid hydrogel particles.

9. The support medium of claim 1, in a form of a slurry, wherein a volume ratio of said plurality of hybrid hydrogel particles to an excess of said dispersion medium ranges from 1:0.001 to 1:3.

10. A process of preparing the support medium of claim 1, comprising:
i. forming a calcium-alginate hydrogel in the presence of said non-alginate soluble polymer to thereby afford a hybrid hydrogel;
ii. pulverizing said hybrid hydrogel to thereby obtain a plurality of hybrid hydrogel particles;
iii. washing said plurality of hybrid hydrogel particles to thereby obtain a plurality of washed hybrid hydrogel particles; and
iv. contacting said washed hybrid hydrogel particles with a solution of gluconate.

11. The process of claim 10, further comprising:
v. draining said hybrid hydrogel particles from excess of said solution of said gluconate to thereby obtain drained hybrid hydrogel particles having gluconate therein.

12. The process of claim 11, further comprising:
vi. contacting said drained hybrid hydrogel particles or non-drained hydrogel particles with a solution of said non-alginate soluble polymer.

13. The process of claim 10, wherein said solution of said gluconate comprises sodium gluconate at a concentration of at least 2 mM.

14. The process of claim 10, wherein said forming said calcium-alginate hydrogel is effected in the presence of an insoluble calcium salt and an acidifier.

15. The process of claim 14, wherein said acidifier is a glucono-δ-lactone.

16. The process of claim 14, wherein said insoluble calcium salt is calcium carbonate.

17. The process of claim 10, wherein a concentration of said non-alginate soluble polymer in Step (i) ranges 0.001-20% (w/v).

18. The process of claim 12, wherein a concentration of said non-alginate soluble polymer ranges 0.0001-4% (w/v).

19. The process of claim 10, wherein said non-alginate soluble polymer is xanthan gum.

20. A selling unit comprising the support medium of claim 1.

21. The selling unit of claim 20, wherein the support medium is sterile and/or detoxified.

22. The selling unit of claim 20, wherein the support medium is ready for use without further dilution.

23. The selling unit of claim 20, wherein the support medium is in the form of a drained slurry.

24. The selling unit of claim 23, further comprising a dilution solution.

25. The selling unit of claim 20, packaged in a packaging material and identified in print on or in said packaging material, for use as a support medium in 3D printing process and/or printer.
